(19) United States
(12) Patent Application Publication
(10) Pub. No.: US 2007/0207989 A1
(43) Pub. Date: Sep. 6, 2007

(54) DITERPENE DERIVATIVES FOR THE TREATMENT OF CARDIOVASCULAR, CANCER AND INFLAMMATORY DISEASES

(75) Inventors: Inderjit Kumar Dev, Durham, NC (US); Ven Subbiah, Greenville, NC (US)

Correspondence Address: PASSE' INTELLECTUAL PROPERTY, LLC, 1717 BRASSFIELD RD., RALEIGH, NC, 27614, US

(73) Assignee: SaviPu Pharmaceuticals, Durham, NC (US)

(21) Appl. No.: 11/681,109

(22) Filed: Mar. 1, 2007

Related U.S. Application Data
(60) Provisional application No. 60/779,142, filed on Mar. 3, 2006.

(51) Int. Cl. A61K 31/58; C07D 307/77

(52) U.S. Cl. 514/176; 514/172; 540/107; 540/120; 540/2; 540/48; 540/51; 540/56; 540/58

(57) ABSTRACT

The present invention relates to useful diterpenes and pharmaceutical compositions containing them of the formula:

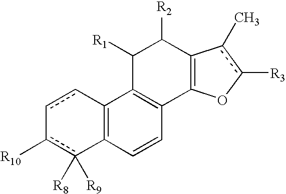

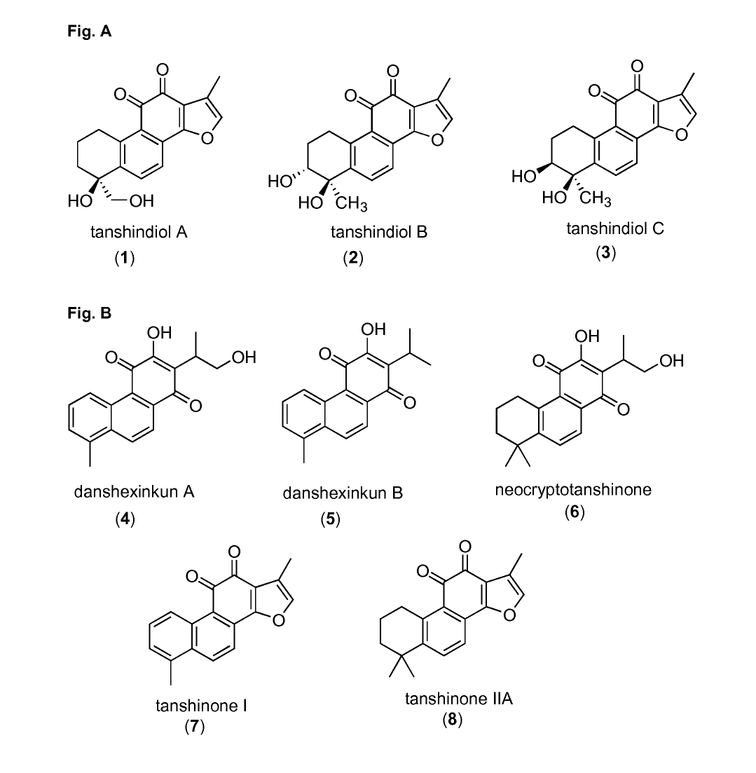

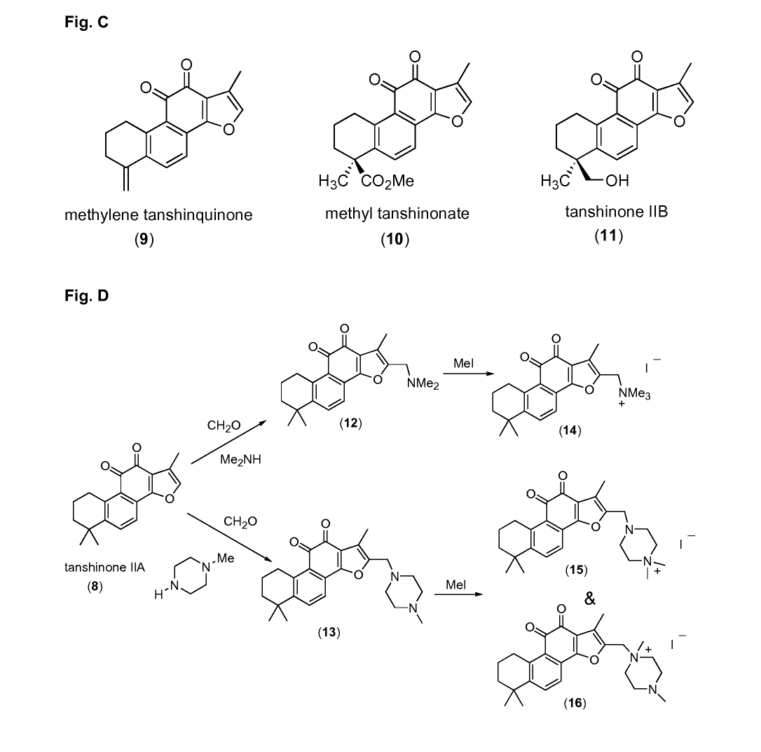

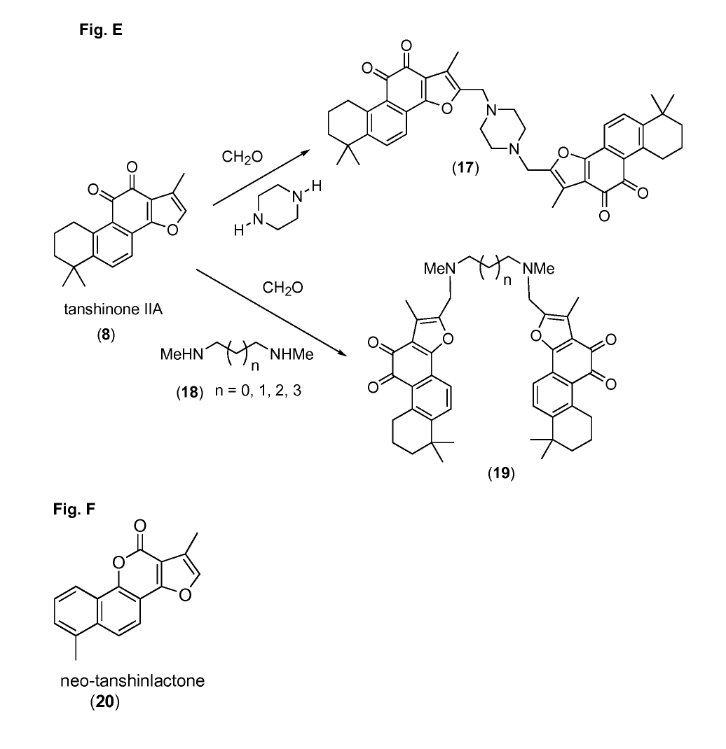

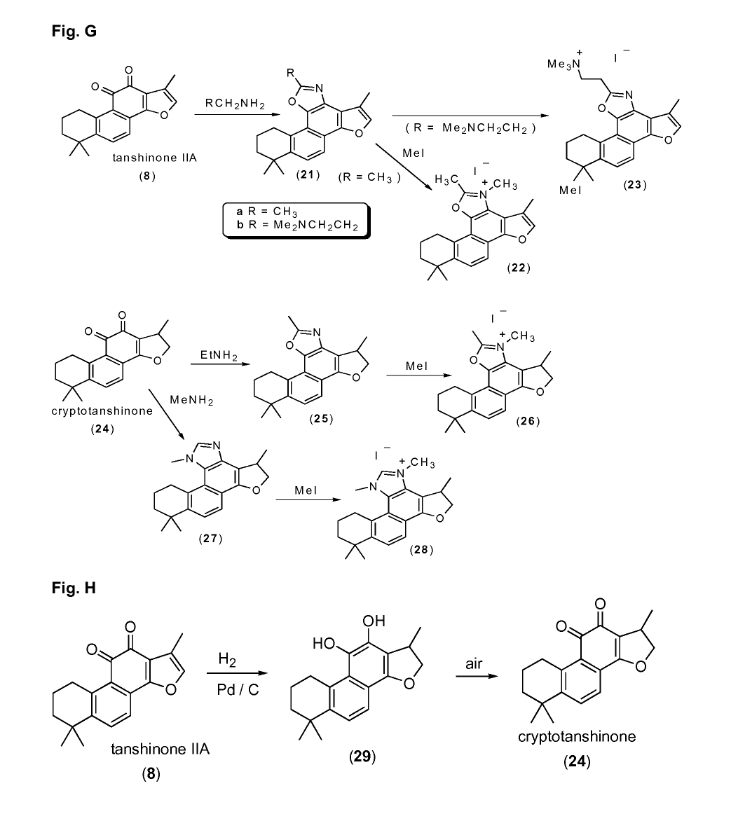

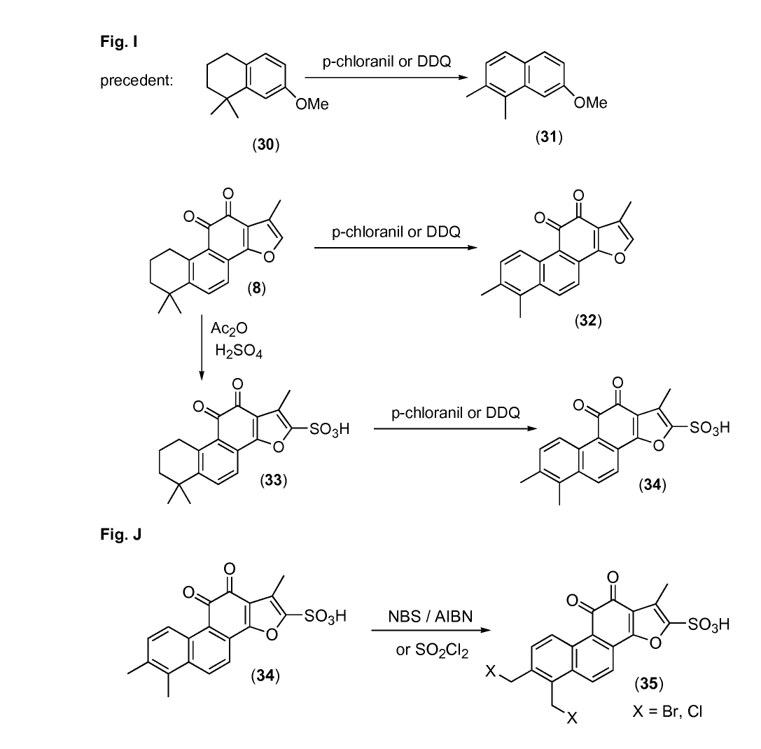

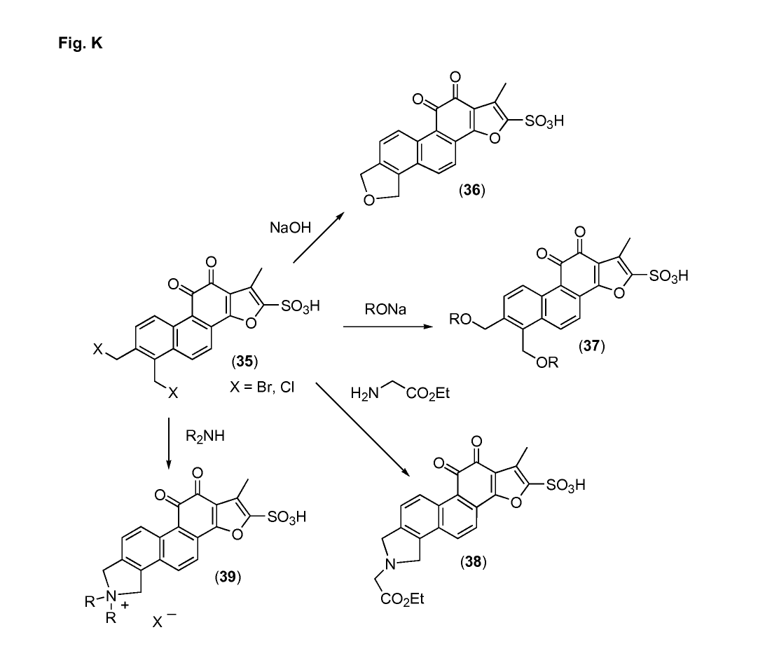

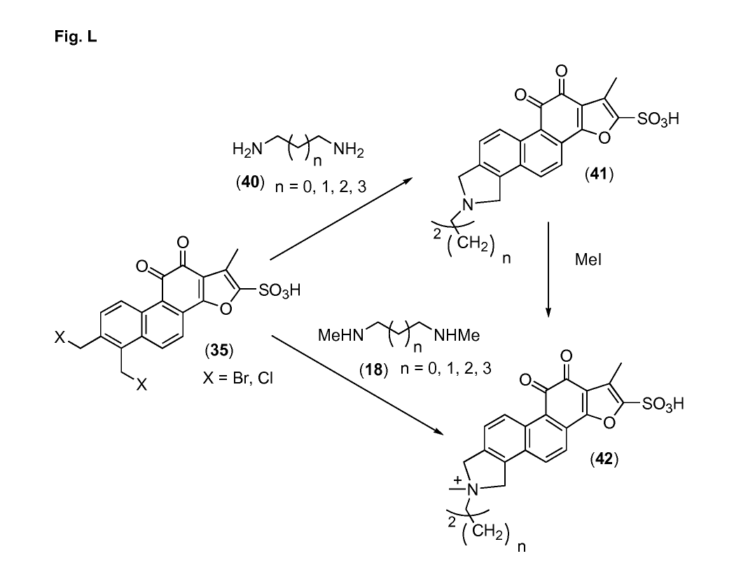

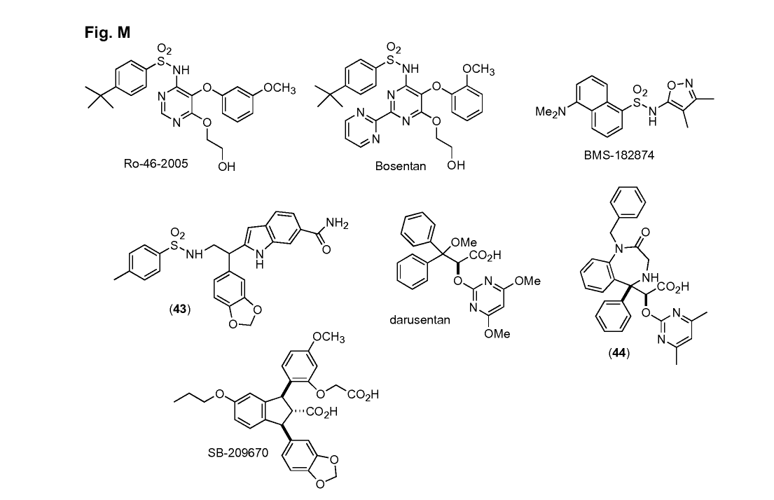

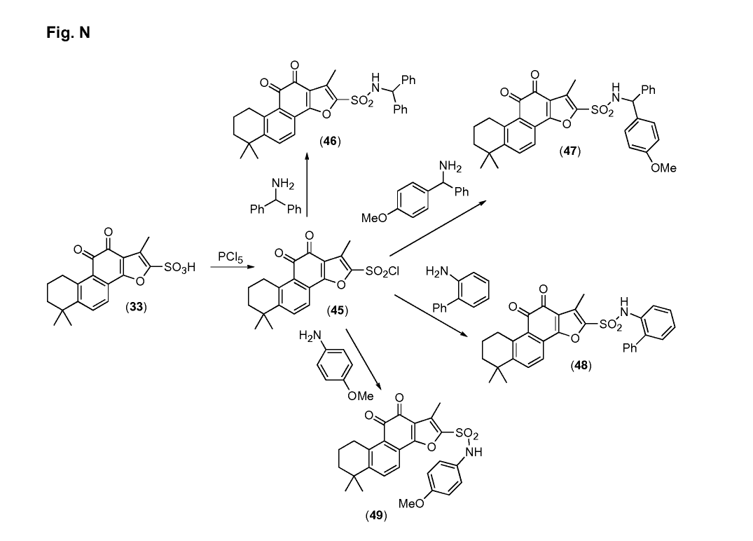

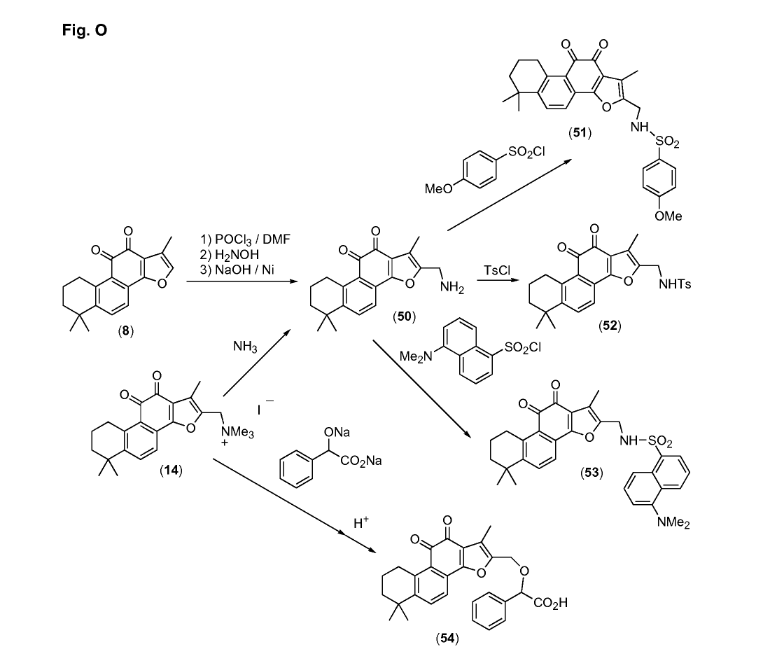

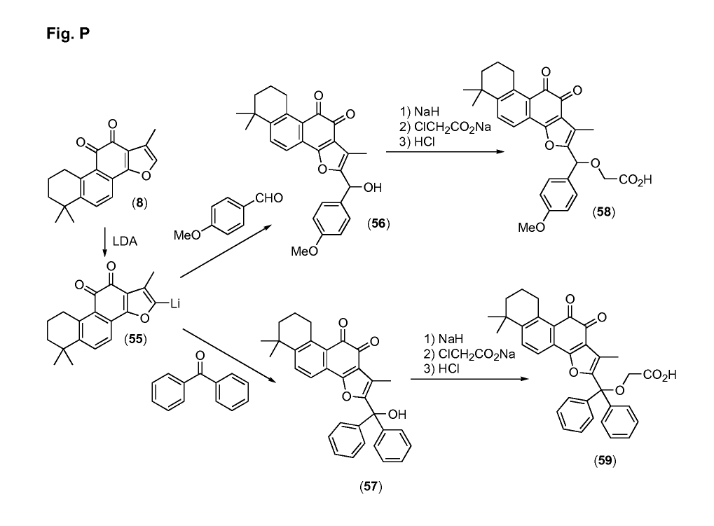

for use in the treatment of cardiovascular and inflammatory diseases and for cancers susceptible to an NF-κB inhibitor and an endothelin receptor inhibitor. The present invention also relates to compounds and methods useful to inhibit cell proliferation and for the induction of apoptosis.

This application claims priority of U.S. Application Ser. No. 60/779,142 filed on Mar. 03, 2006 and incorporated herein in its entirety by reference.

### BACKGROUND OF THE INVENTION

1. Field of the invention

This invention relates to the use of certain novel diterpenes which are inhibitors of nuclear factor kappa B (NF-κB) and inhibit the activity of the endothelin receptor. In particular, it relates to useful diterpenes and pharmaceutical compositions containing them for use in the treatment of cardiovascular and inflammatory diseases and for cancers susceptible to an NF-κB inhibitor and an endothelin receptor inhibitor. The present invention also relates to compounds and methods useful to inhibit cell proliferation and for the induction of apoptosis.

2. Description of the Related Art

Endothelin is a vasoconstrictor peptide composed of 21 amino acids and derived in mammals from the endothelium. These endothelin receptors exist in various tissue and organs such as vessels, trachea and the like and their excessive stimulation can lead to circulatory diseases such as pulmonary hypertension, acute and chronic heart failure, acute and chronic renal failure, atherosclerosis, cerebrovascular diseases and the like.

NF-κB is one of the principal inducible transcription factors in mammals and has been shown to play a pivotal role in the mammalian innate immune response and chronic inflammatory conditions (Jour. Pharm. and Phar. 2002, 54: 453-472). The signaling mechanism of NF-κB involves an integrated sequence of protein-regulated steps and many are potential key targets for intervention in treating certain NF-κB cascade dependant inflammatory conditions and cancers.

More specifically, the family of NF-κB transcription factors comprises important regulatory proteins that impact virtually every feature of cellular adaptation, including responses to stress, inflammatory reactions, activation of immune cell function, cellular proliferation, programmed cell death (apoptosis), differentiation and oncogenesis (1). NF-κB regulates more than 150 genes, including cytokines, chemokines, cell adhesion molecules, and growth factors (2). It is therefore not surprising that diseases result when NF-κB -dependent transcription is not appropriately-regulated. NF-κB has been implicated in several pathologies, including certain cancers (e.g., Hodgkin's disease, breast cancer, and prostate cancer), diseases associated with inflammation (e.g., rheumatoid arthritis, asthma, inflammatory bowel disease (e.g., Crohn's disease and ulcerative colitis), alcoholic liver disease, non-alcoholic steatohepatitis, pancreatitis, primary dysmenorrhea, psoriasis, and atherosclerosis) and Alzheimer's disease. Several mediators of inflammation are under the influence of activated NF-κB including inducible nitric oxide synthase, the subsequent production of nitric oxide and prostaglandin synthase. It has further been shown that compounds which interfere with COX-2 act via the inhibition of NF-κB. NF-κB consists of different combinations of Rel proteins in various heterodimers and homodimers and has previously been represented by the subunits p65/p50. All the Rel proteins share a conserved region of 300 amino acids at the N-terminal responsible for DNA-binding, dimerisation and interaction with the NF-κB inhibitory protein I-kappaB. NF-κB is responsible in several signaling cascades and the two most important of which are ones associated with mammalian immune response of the interleukin/lipopolysaccharide pathway. There are pathways involved with NF-κB that are critically involved in apoptosis. NF-κB binding by RelA is constituitively elevated in human metastatic melanoma cultures relative to normal melanocytes.

NF-κB is a collective name for dimeric transcription factors comprising the Rel family of DNA-binding proteins (3, 4). All members of this family are characterized by the presence of a conserved protein motif called the Rel homology domain (RHD) that is responsible for dimer formation, nuclear translocation, sequence-specific DNA recognition and interaction with inhibitory proteins collectively known as I-κB. Any homodimer or heterodimer combination of family members constitutes NF-κB.

### Regulation of NF-κB Activity

The activity of NF-κB is regulated through an assortment of complex signaling pathways. NF-κB is negatively regulated through interaction with I-KB (5). Each I-KB possesses an N-terminal regulatory domain for a signal dependent I-KB proteolysis, a domain composed of six or seven ankyrin repeats to mediate interaction with the Rel proteins, and a C-terminal domain containing a PEST motif that is implicated in constitutive I-κB turnover. Inactive forms of NF-κB reside in the cytoplasm as NF-κB/I-κB complexes, because I-κB binding to NF-κB blocks the ability of the nuclear import proteins to recognize and bind to the nuclear localization signal in the RHD.

NF-κB activation occurs when NF-κB is translocated to the nucleus following its release from I-κB. I-κB dissociation arises through its phosphorylation by an inducible I-κB kinase (IKK) and ubiquitination by I-κB ubiquitin ligase, which flags it for proteolysis by the 26S proteosome. Since the ubiquitin ligase and the 26S proteosome are constitutively expressed, the de-repression of NF-κB functional activity is largely governed by those signals that induce the expression of IKK, which include inflammatory cytokines, mitogens, viral proteins, and stress.

IKK is also known as the signalsome, which consists of a large multi-subunit complex containing the catalytic subunits IKK.alpha./IKK-1 and IKK.beta./IKK-2, a structural subunit termed NF-κB essential modulator (NEMO), as well as perhaps other components (6, 7). NEMO, also known as IKK.γ. and IKKAP-1, functions as an adapter protein to permit communication between the catalytic subunits and upstream activators (7). Activation of NF-κB is a tightly controlled process and cannot occur without NEMO (8, 9).

Protein phosphorylation positively regulates NF-κB activity (1). Protein phosphorylation enhances the transcriptional activity of NF-κB, presumably through the phosphorylated protein's interaction with other transcriptional co-activators. Protein kinase A (PKA), casein kinase 11 (CKII), and p38 mitogen-activated protein kinase (MAPK) have been implicated in the phosphorylation of NF-κB.

The activity of NF-κB is also subject to autoregulatory mechanisms to ensure that NF-κB -dependent transcription is coordinately-linked to the signal-inducing response. For example, the I-κB genes contain NF-κB binding sites within their promoter structures that result in their increased transcription upon NF-κB binding. The expressed I-κB proteins migrate into the nucleus to bind the NF-κB and mediate transport of NF-κB to the cytoplasm where it remains inactive.

### Role of NF-κB in Disease and Disorders

NF-κB contributes to progression of cancers by serving both as positive regulators of cell growth and as a negative regulator of apoptosis (10, 11). NF-κB stimulates expression of cell cycle-specific proteins c-Myc and cyclin D1 (12, 13). The constitutive expression of these proteins results in sustained cell proliferation. Continued expression of c-Myc ultimately leads to apoptosis. NF-κB can block c-Myc's apoptosis effects, thereby stimulating proliferation without cytotoxicity. NF-κB also inhibits the ability of Tumor Necrosis Factor (TNF) to induce cell death as well as protect cells from the effects of ionizing radiation and chemotherapeutic drugs (14). Thus, NF-κB promotes both hyperplasia and resistance to oncological treatments, which are hallmarks of many cancers.

Inhibition of NF-κB activation has been linked to the chemopreventive properties of several anti-cancer compounds (e.g., selenium, flavonoids, etc.) (15, 16). Although long-term inhibition could have unwanted effects on immune response, down-regulation of NF-κB activity is considered a very attractive strategy for developing new cancer treatments.

Recently, Shen et al. demonstrated that certain oligonucleotides that contain polyguanonsines are potent inhibitors of the proliferation of murine prostate cancer cells (17). The specific DNA-binding activities of NF-κB and another transcription factor, AP-1 were reduced in cells treated with these oligonucleotides. Oligonucleotides displaying antiproliferative effects were capable of forming higher order structures containing guanosine-quartets (G-quartets). The requirement of G-quartets for inducing apoptosis was suggested by experimental observations wherein mutations that destroyed the capacity to form a G-quartet structure correlated with abolishment of the antitumor activities of the oligonucleotide (17).

In the case of inflammation, NF-κB plays important roles in both the initiation and maintenance of the inflammatory response (1). Activated T cells, such as activated CD₄+ T helper cells, trigger immune inflammation. The T helper cell population can differentiate further to two subset populations that have opposite effects on the inflammatory response. The Th1 subset is considered proinflammatory, as these cells mediate cellular immunity and activate macrophages. The Th2 subset is considered anti-inflammatory, as these cells mediate humoral immunity and down-regulate macrophage activation. The subsets are distinguishable by the different types of cytokine profiles that they express upon differentiation. NF-κB stimulates production of cytokine profiles characteristic of the Th1 subset type, leading to a proinflammatory response. Conversely, suppression of NF-κB activation leads to production of cytokine profiles characteristic of the Th2 subset type that mediates an anti-inflammatory response.

Once activated, these inflammatory cytokines and growth factors can act through autocrine loops to maintain NF-κB activation in non-immune cells within the lesion (1). For example, NF-κB regulates the expression of cytokines Interleukin 1 β (IL-1β) and Tumor Necrosis Factor alpha (TNFα), which are considered essential mediators of the inflammatory response. Conversely, these gene products positively activate NF-κB expression that leads to persistence of the inflammatory state. For example, TNF products have been implicated in promoting inflammation in several gastrointestinal clinical disorders that include: alcoholic liver disease, non-alcoholic steatohepatitis, prancreatitis (including chronic, acute and alcohol-induced), and inflammatory bowel disorders, such as ulcerative colitis and Crohn's Disease.

Continued NF-κB activation also promotes tissue remodeling in the inflammatory lesions (1). Several NF-κB -responsive genes have been implicated in this regard and include growth factors that are important to neovascularization (e.g., VEGF), matrix proteinases (including metalloproteases), cyclooxygenase, nitric oxide synthase, and enzymes that are involved in the synthesis of proinflammatory prostaglandins, nitric oxide, and nitric oxide metabolites (1). Such tissue remodeling is often accompanied by breakdown of healthy cells as well as by hyperplasia, both of which are often observed in rheumatoid arthritis and other inflammatory diseases (1).

Suppression of NF-κB activity alleviates many inflammatory disease conditions and increases the susceptibility of certain cancers to effective treatment. Several anti-inflammatory drugs directly target the NF-κB signaling pathway. Glucocorticoids, one member of the general steroid family of anti-inflammatory drugs, interfere with NF-κB function through the interaction of the glucocorticoid receptor with NF-κB (18). Gold compounds interfere with the DNA-binding activity of NF-κB (19). Aspirin and sodium salicylate, as representatives of non-steroid anti-inflammatory drugs, inhibit IKKβ activity and thereby prevent signal-inducible I-κB turnover (20). Dietary supplements with anti-inflammatory and anti-tumor activities prevent NF-κB activation by interfering with pathways leading to IKK activation. Vitamins C and E, prostaglandins, and other antioxidants, scavenge reactive oxygen species that are required for NF-κB activation (21, 22). Specific NF-κB decoys that mimic natural NF-κB ligands (e.g., synthetic double-stranded oligodeoxynucleotides that contain the NF-κB binding site) can suppress NF-κB activity and prevent recurrent arthritis in animal models (23).

Despite the promise of anti-inflammatory drugs in treating inflammatory diseases, many diseases are non-responsive to these modalities. For example, many patients with chronic inflammatory diseases, such as Crohn's disease, fail to respond to steroid treatment. Recent studies suggest that one basis for the steroid unresponsiveness may be attributed to NF-κB and other NF-κB -responsive gene products antagonizing glucocorticoid receptor expression, which is necessary for the steroid's anti-inflammatory activity (24).

Alzheimer's disease represents another example of a condition that displays an inflammatory component in its pathogenesis. Recent studies indicate that abnormal regulation of the NF-κB pathway may be central to the pathogenesis of Alzheimer's disease. NF-κB activation correlates with the initiation of neuritic plaques and neuronal apoptosis during the early phases of the disease. For example, NF-κB immunoreactivity is found predominantly in and around early neuritic plaque types, whereas mature plaque types display reduced NF-κB activity (25).

These data suggest that NF-κB and endothelin receptor are two promising and valid molecular targets for the treatment of cancer, inflammatory and cardiovascular diseases. The inventors believe that the presence of endothelin receptor and NF-κB antagonistic activity on the same molecule can be synergistic due to several reasons. First, reductions in endothelin levels due to the inhibition of gene transcription by NFKB will make inhibition of endothelin receptor more effective. Most endothelin receptor antagonists compete with endothelin for receptor binding; thus inhibition of endothelin receptor antagonists in the presence of reduced concentrations of endothelin should be enhanced substantially. Second, the effects of endothelin receptor antagonists and NF-κB antagonists on the apoptotic pathways complement each other. The inhibition of NF-κB induces apoptosis by regulating gene transcription of anti-apoptotic genes; whereas, endothelin acts as an antiapoptotic factor, modulating cell survival pathways through Bcl-2 and phosphatidylinositol 3-kinase/Akt pathways.

In cancer, multi-targeted molecular therapy can provide several benefits including the ability to overcome resistance to cancer chemotherapeutic agents and also have a broad spectrum of activity for many different hard-to-treat cancers such as those of the prostate, breast, lung, colon, ovarian and melanoma. Moreover, the potential synergistic interaction due to simultaneous inhibition of two key cellular pathways could also provide additional benefits to cancer patients. Pulmonary arterial hypertension (PAH) is a progressive disease that is usually fatal within 3 years, if untreated. PAH is characterized by obstructive vascular remodeling and vasoconstriction leading to right-sided heart failure. The combined inhibition of the NFκB and endothelin receptor could effectively block both the vasoconstriction and the vascular remodeling and provide effective treatment for PAH.

Accordingly it would be extremely useful to find compositions which can inhibit both endothelin and NF-κB.

### SUMMARY OF THE INVENTION

Certain novel compositions represented by formula I have been discovered to inhibit endothelin receptor activity and also inhibit NF-κB and are thus useful to treat certain conditions not previously known to be susceptible to treatment with an endothelin antagonist alone or an NF-κB inhibitor alone and also provide additional benefits to the patients due to the potential synergistic interaction between the inhibitors of the two key cellular pathways.

One embodiment of the invention is a composition of the formula

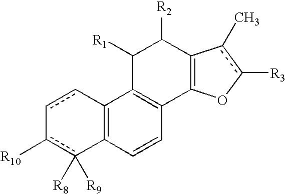

wherein R₁ and R₂ each being the same or different are O or hydroxyl or wherein R₁ and R₂ are taken together to form a 5 member ring of the formula:

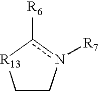

wherein R₆ and R₇ each being the same or different are hydrogen or alkyl of 1 to 6 carbon atoms; wherein R₃ is hydrogen, (CH3)₂N(alkyl of 1 to 6 carbon atoms), R₄—N—R₄—N—R₅, SO₂R₁₁, or a composition of the formula:

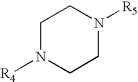

wherein R₄ is alkyl of 1 to 6 carbon atoms, R₅ is hydrogen alkyl of 1 to 6 carbon atoms, or methyl tashione IIA and R₁₁ is hydroxyl, diphenyl-NH, alkoxy of 1 to 6 carbon atom phenyl R₁₂—NH, or phenyl substituted phenyl (R₁₂)—NH and wherein R₁₂ is alkyl of 0 to 6 carbon atoms, phenyl substituted alkyl or alkyl of 1 to 6 carbon atoms-COOH;
wherein R₈, R₉ and R₁₀ separately or together is hydrogen, alkyl of 1 to 6 carbon atoms or haloalkyl of 1 to 6 carbon atoms; or wherein R₈ and R₉ are taken together to for a ring of the formula:

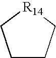

wherein R₁₃ and R₁₄ each being the same or different are O, N Alkyl of 1-6 carbon atoms-N, Dialklyl of 1-6 carbon atoms-N or (alkyl of 1 to 6 carbon atoms)-CO₂-(alkyl of 1 to 6 carbon atoms) wherein when R₃ is hydrogen R₁ and R₂ can not both be O.

And another embodiment relates to a composition according to the above embodiments wherein the composition is used to treat a disease selected from the group consisting of inflammatory and tissue repair disorders, particularly rheumatoid arthritis, inflammatory bowel disease, asthma and chronic obstructive pulmonary disease, osteoarthritis, osteoporosis and fibrotic diseases, dermatosis, including psoriasis, atopic dermatitis and ultraviolet radiation—induced skin damage, autoimmune diseases including systemic lupus eythematosus, multiple sclerosis, psoriatic arthritis, alkylosing spondylitis, tissue and organ rejection, Alzheimer's disease, stroke, atherosclerosis, restenosis, diabetes, glomerulonephritis, cancer, including Hodgkins disease, cachexia, inflammation associated with infection and certain viral infections, including aquired immune deficiency syndrome, adult respiratory distress syndrome, and Ataxia Telangiestasia.

Other embodiments of the invention will be clear from the discovery that the compounds of the invention posses both Endothelin receptor and NF-κB inhibitory activity and are therefore useful for the treatment of disease other than possible with just endothelin antagonist activity previously known.

### BRIEF DESCRIPTION OF THE DRAWINGS

FIGS. A through P show the production methods of the compositions of this invention.

### DETAILED DESCRIPTION OF THE INVENTION

This invention provides methods for treating a variety of diseases associated with endothelin receptor pathway and NF-κB activation including cancer, cardiovascular diseases and inflammatory and tissue repair disorders; particularly rheumatoid arthritis, inflammatory bowel disease, asthma and COPD (chronic obstructive pulmonary disease) osteoarthritis, osteoporosis and fibrotic diseases; dermatosis, including psoriasis, atopic dermatitis and ultraviolet radiation (UV)-induced skin damage; autoimmune diseases including systemic lupus eythematosus, multiple sclerosis, psoriatic arthritis, alkylosing spondylitis, tissue and organ rejection, Alzheimer's disease, stroke, atherosclerosis, restenosis, diabetes, glomerulonephritis, cancer, including Hodgkins disease, cachexia, inflammation associated with infection and certain viral infections, including aquired immune deficiency syndrome (AIDS), adult respiratory distress syndrome, and Ataxia Telangiestasia.

The present invention includes all hydrates, solvates, complexes and prodrugs of the compounds of this invention. Prodrugs are any covalently bonded compounds, which release the active parent, drug according to Formula I in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether or not existing in predominantly one form.

The meaning of any substituent at any one occurrence in Formula I or any subformula thereof is independent of its meaning, or any other substituent's meaning, at any other occurrence, unless specified otherwise.

As used herein, “metabolic ester residue” refers to an ester residue which decomposes to reproduce carboxylic acids in a living body. See for example U.S. Pat. No. 5,248,807 which describes metabolic ester residue triterpene derivatives.

This invention provides a pharmaceutical composition, which comprises a compound according to Formula I and a pharmaceutically acceptable carrier, diluent or excipient. Accordingly, the compounds of Formula I may be used in the manufacture of a medicament. Pharmaceutical compositions of the compounds of Formula I prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filing for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

Typical compositions for inhalation are in the form of a dry powder, solution, suspension or emulsion. Administration may for example be by dry powder inhaler (such as unit dose or multi-dose inhaler, or by nebulisation or in the form of a pressurized aerosol. Dry powder compositions typically employ a carrier such as lactose, trehalose or starch. Compositions for nebulisation typically employ water as vehicle. Pressurized aerosols typically employ a propellant such as dichlorodifluoromethane, trichlorofluoromethane or, more preferably, 1,1,1,2-tetrafluoroethane, 1,1,1,2,3,3,3-heptafluoro-n-propane or mixtures thereof. Pressurized aerosol formulations may be in the form of a solution (perhaps employing a solubilising agent such as ethanol) or a suspension which may be excipient free or employ excipients including surfactants and/or co-solvents (e.g. ethanol). In dry powder compositions and suspension aerosol compositions the active ingredient will preferably be of a size suitable for inhalation (typically having mass median diameter (MMD) less than 20 microns, e.g., 1-10 especially 1-5 microns). Size reduction of the active ingredient may be necessary, e.g., by micronisation.

Typical compositions for nasal delivery include those mentioned above for inhalation and further include non-pressurized compositions in the form of a solution or suspension in an inert vehicle such as water optionally in combination with conventional excipients such as buffers, anti-microbials, tonicity modifying agents and viscosity modifying agents which may be administered by nasal pump.

For rectal administration, the compounds of this invention may also be combined with excipients such as cocoa butter, glycerin, gelatin or polyethylene glycols and molded into a suppository.

The methods of the present invention include topical inhaled and intracolonic administration of the compounds of Formula I. By topical administration is meant non-systemic administration, including the application of a compound of the invention externally to the epidermis, to the buccal cavity and instillation of such a compound into the ear, eye and nose, wherein the compound does not significantly enter the blood stream. By systemic administration is meant oral, intravenous, intraperitoneal and intramuscular administration. The amount of a compound of the invention (hereinafter referred to as the active ingredient) required for therapeutic or prophylactic effect upon topical administration will, of course, vary with the compound chosen, the nature and severity of the condition being treated and the animal undergoing treatment, and is ultimately at the discretion of the physician.

While it is possible for an active ingredient to be administered alone as the raw chemical, it is preferable to present it as a pharmaceutical formulation. The active ingredient may comprise, for topical administration, from 0.01 to 5.0 wt % of the formulation.

The topical formulations of the present invention, both for veterinary and for human medical use, comprise an active ingredient together with one or more acceptable carriers therefore and optionally any other therapeutic ingredients. The carrier must be “acceptable” in the sense of being compatible with the other ingredients of the formulation and not deleterious to the recipient thereof.

Formulations suitable for topical administration include liquid or semi-liquid preparations suitable for penetration through the skin to the site of where treatment is required such as: liniments, lotions, creams, ointments or pastes, and drops suitable for administration to the eye, ear or nose.

### Utility of the Present Invention

The compounds of Formula I are useful as inhibitors of NF-κB activation. The present method utilizes compositions and formulations of said compounds, including pharmaceutical compositions and formulations of said compounds. The present invention particularly provides methods of treatment of diseases associated with inappropriate NF-κB activation, which methods comprise administering to an animal, particularly a mammal, most particularly a human in need thereof one or more compounds of Formula I. The present invention particularly provides methods for treating inflammatory and tissue repair disorders, particularly rheumatoid arthritis, inflammatory bowel disease, asthma and COPD (chronic obstructive pulmonary disease); osteoarthritis, osteoporosis and fibrotic diseases; dermatosis, including psoriasis, atopic dermatitis and ultraviolet radiation (UV)-induced skin damage, autoimmune diseases including systemic lupus eythematosus, multiple sclerosis, psoriatic arthritis, alkylosing spondylitis, tissue and organ rejection, Alzheimer's disease, stroke, atherosclerosis, restenosis, diabetes, glomerulonephritis, cancer, including Hodgkins disease, cachexia, inflammation associated with infection and certain viral infections, including aquired immune deficiency syndrome (AIDS), adult respiratory distress syndrome and Ataxia Telangiestasia.

For acute therapy, parenteral administration of one or more compounds of Formula I is useful. An intravenous infusion of the compound in 5% dextrose in water or normal saline, or a similar formulation with suitable excipients, is most effective, although an intramuscular bolus injection is also useful. Typically, the parenteral dose will be about 0.01 to about 50 mg/kg; preferably between 0.1 and 20 mg/kg, in a manner to maintain the concentration of drug in the plasma at a concentration effective to inhibit activation of NF-κB. The compounds are administered one to four times daily at a level to achieve a total daily dose of about 0.4 to about 80 mg/kg/day. The precise amount of a compound used in the present method which is therapeutically effective, and the route by which such compound is best administered, is readily determined by one of ordinary skill in the art by comparing the blood level of the agent to the concentration required to have a therapeutic effect.

### Examples and Preparation of Novel Compositions

### Chemical modifications of PM2010 (Tanshinone IIA)

We have recently found that usable quantities of tanshinone IIA 8 can be isolated from clary sage (Salvia sclarea L.). Although 8 demonstrated a modest inhibition of endothelin receptor A (ETA), NF-κB and tumor cell lines, our goal is to make derivatives of this compound which are expected to have greater activity, and to assess their effectiveness in inhibition of ETA and activation of NF-κB. Several diterpenoids including 8 block NFκB from binding to κB consensus DNA and inhibit tumor cell lines.

Many diterpenoids isolated from the roots of Salviae Miltiorrhizae Radix (“Danshen”) inhibit cell proliferation of a number of carcinoma cell lines. Of the 15 compounds screened in this study, three of the most potent are those shown in FIG. A, these compounds having ID₅₀ values ranging from 0.60 to 3.39 μg/mL when tested against four different cell lines.

The least potent of the compounds tested were those that lacked the furan and o-quinone rings, compounds 4-6 illustrated in FIG. B. This suggests that these structural features (the furan ring and/or o-quinone) are important for activity. Two derivatives that do possess these structural features, yet still lack significant activity are tanshinone 17 and tanshinone IIA 8, also shown in FIG. B.

One might be inclined to believe that the presence of hydroxyl groups in ring A is necessary for activity, were it not for the following observations: methylene tanshinquinone 9 (FIG. C) is more potent than any of the tanshindiols (compounds 1, 2 and 3), and methyl tanshinonate 10 is equally potent as the tanshindiols (at least with respect to two of the four cell lines tested). Furthermore, tanshinone IIB 11 showed very little activity-about on a par with tanshinone IIA 8. Nevertheless, it does appear that the presence of hydroxyl groups in the A ring has a favorable effect on activity.

It is also interesting to note that the results of Wu and coworkers tend to contradict computation-based results of Luo, et al. Luo's results suggest that compounds with an aromatic ring A are more potent than those with an alicyclic ring A, as were those with a reduced furan ring (ring D) compared to those with an aromatic furan ring. The data reported by Wu, however, seems to indicate just the opposite-those with an alicyclic ring A are more potent, as are those with an aromatic ring D. This, coupled with a lack of a precise understanding of the binding and mode of action of such compounds leave structure/activity relationships still somewhat poorly understood, despite much work in the area.

Approach: Since the presence of hydroxyl groups on tanshinone derivatives seems to increase their potency against various cancer cell lines, we intend to prepare derivatives substituted with highly electrophilic sites in an effort to increase the activity of such compounds. It is not clear why the hydroxyl groups in compounds 1-3 increase the activity of these compounds relative to the non-hydroxylated congeners-they could be interacting with NF-κB and block binding to consensus B DNA, they could be increasing the affinity of such compounds for the anionic phosphate “backbone” of DNA, or they could be playing some other role. While knowing the precise role played by the hydroxyl groups in compounds 1-3 would be helpful, we can nevertheless speculate that the incorporation of highly electrophilic sites into tanshinone IIA derivatives might serve a similar purpose, whatever it might be. Thus, derivatives proposed below incorporate additional sites for hydrogen bonding, cationic sites, or both.

Our target molecules can be grouped into two broad categories-those where modification is made in ring A thereby having the closest structural similarity to compounds 1-3), and those where modifications are made elsewhere operating under the assumption that a specific binding to NF-B/DNA complexes is not involved, and any increase in the molecule's general polarity and/or electrophilicity may increase activity by increasing the accumulation of targeted compounds in nucleus. Accumulation of many DNA-targeted drugs in nucleus is common. Synthesis of the latter group is more highly precedented and should proceed in relatively few steps, and will be discussed first.

Synthesis: Condensation of secondary amines and formaldehyde with tanshinone IIA 8 via a Mannich reaction has previously been reported, and yields aminomethylated derivatives such as 12 and 13 (FIG. D).

We intend to allow these compounds to react with alkylating agents such as methyl iodide to yield quaternary ammonium salts 14 and 15, respectively. (Compound 15 may be contaminated with some of the isomeric compound 16, but we expect 15 to be the major isomer due to lesser amounts of steric hindrance around the methylated nitrogen of 13. Compounds such as 15 are expected to be more stable than either 14 or 16 since they will not be as susceptible to elimination of the ammonium salt moiety.

As with all compounds in this project, initial assays will guide further synthetic efforts. Thus, if salts 14 and 15 prove to be stable and have favorable biological activity, other derivatives will be prepared. Both the nature of the secondary amine and the nature of the quaternizing agent could be easily varied. Examples of other readily available secondary amines that would be tested include morpholine, pyrrolidine, piperidine and diethylamine. Ethyl iodide and chloroacetic acid (the use of which would result in the formation of a zwitterion) are examples of other alkylating agents that could be investigated. In those cases where ammonium salts such as 14 and 15 are initially prepared as iodide salts, exchange of the iodide counterion for more biologically relevant ions such as phosphate or citrate will most likely precede any biological testing.

Use of piperazine as the secondary amine in a Mannich reaction of 8 with formaldehyde should allow synthesis of dimeric species 17 (FIG. E). Since it is known that linkage of two planar groups (capable of DNA intercalation) by aminoalkyl “spacer” groups often increases effectiveness of anticancer compounds, compounds such as 17 would be of much interest. N,N′-dimethylaminoalkanes 18 of varying chain lengths would be used to probe the effects of “spacer” length on cytotoxicity of derivatives 19. As with the Mannich products 12 and 13, alkylation with methyl iodide would lead to methiodide salts which would also be tested for biological activity.

Modification of the quinone functionality on tanshinone IIA will also be investigated. Though presence of an o-quinone appears to be beneficial for activity, once again its role is not precisely known-whether it is needed as an electron acceptor, or simply to provide a polar group while maintaining the planarity of the molecule. Support for the idea that an o-quinone per se is not required for activity can be found in the observation that neo-tanshinlactone 20 (FIG. F) has proven very effective against several human cancer cell lines, particularly breast cancer, where in some cases it was found to be ten times more potent and twenty times more selective than tamoxifen citrate.

The derivatives proposed below retain the planarity, and in several cases, the electron accepting ability of the o-quinone functionality, yet, like salts 14 and 15, are expected to have increased water solubility and electrophilicity owing to the presence of a quaternary salt.

Both tanshinone IIA 8 and its reduced form cryptotanshinone 24 have been shown to form oxazoles 21a and 25 respectively, when allowed to react with ethylamine (FIG. G). Reaction of 8 and 24 with a few other primary amines to give the corresponding oxazoles has also been reported). Analogously to the proposed work described above, we intend to convert such compounds into the corresponding methylated salts 22 and 26 to increase their water solubility and electrophilicity. Methylation of benzoxazoles to give products such as these is a known procedure (. Preparation of 21b and its conversion to 23 should proceed in an analogous manner (methylation is expected at the more nucleophilic aliphatic nitrogen rather than the oxazole nitrogen).

As before, if such compounds demonstrate promising cytotoxicity, numerous analogs incorporating other primary amines could be prepared and evaluated.

When cryptotanshinone 24 was allowed to react with methylamine, however, instead of obtaining the corresponding oxazole, imidazole derivative 27 was obtained in 37% yield. Methylation of this compound should be even more facile than methylation of 25, and would be expected to produce methiodide salt 28. To our knowledge, treatment of tanshinone IIA 8 with methylamine has not been carried out, and so it is uncertain as to whether the analogous oxazole or imidazole would be obtained in this reaction. Either product however could be methylated and would be of interest.

Though initial work is likely to involve derivatization of tanshinone IIA, compounds 26 and 28 could still be prepared from this starting material, since conversion of tanshinone IIA 8 into cryptotanshinone 24 could be achieved by the reaction sequence depicted in FIG. H.

Reduction of 8 by catalytic hydrogenation is expected to yield 29, as this reagent has previously been shown to reduce benzofurans to 2,3-dihydrobenzofurans in good yield. Conversion of the quinone to the corresponding hydroquinone is expected to accompany this reduction, however, and so oxidation back to quinone 24 will be required. Such oxidations are extremely facile, however, often occurring simply by exposure to air.

As mentioned earlier, a second part to the proposed work involves modification of the A ring of tanshinone IIA 8. As direct, controlled fictionalization of the alicyclic ring would be very difficult, the plan is to first aromatize the ring by treatment with DDQ or p-chloranil (FIG. I). Such a reaction is expected to involve a concomitant methyl shift, yielding 32, in direct analogy to the reported preparation of 31 from 30 by treatment with the same reagents. If the furan ring of 8 proves to be sensitive to these oxidative conditions, it may be necessary to reduce its nucleophilicity by the addition of an electron withdrawing group. This could easily be accomplished by treatment with sulfuric acid and acetic anhydride to give sulfonic acid derivative 33, in a well-precedented reaction. Compound 33 could then be transformed into 34 by the method just described.

Compound 34 would have the additional advantage that its sodium salt would be expected to have greatly enhanced water solubility, which, as mentioned earlier, is a favorable quality. Further proposed reactions will be shown using derivative 34, with the understanding that should such “protection” be found unnecessary, similar reactions could also be carried out on 32.

Conversion of 1,2-dimethylnaphthalene derivatives to the corresponding halomethyl compounds has been reported to take place using either NBS (for the introduction of bromine) or sulfuryl chloride (for the introduction of chlorine), and so these same reagents are proposed for the synthesis of compound 35 (FIG. J) Although halogenation of the furan methyl is a possible side reaction, it is expected to be less favored due to steric hindrance considerations.

Compound 35 would be the common intermediate for the synthesis of a number of functionalized derivatives, shown in FIG. K. Simple treatment with hydroxide would lead to 36, the least polar, but also the least sterically demanding of the derivatives proposed. A similar reaction with a series of alkoxides would lead to diethers 37. Alkoxides investigated would range from simple ones (such as methoxide and ethoxide), to more complex alkoxides derived from oxygenated alcohols, such as 2-methoxyethanol and di(ehtylene glycol) monomethyl ether. These latter compounds would be expected to have increased water solubility, thereby improving their bioavailability. Reaction with ethyl glycinate would lead to pyrrolidine derivative 38, and further fictionalization of this compound could take place via hydrolysis of the ethyl ester to the corresponding carboxylic acid. Similarly, reaction of 35 with secondary amines would lead to ammonium salts 39. As before, a series of common secondary amines would be investigated, such as dimethylamine, diethylamine, morpholine, pyrrolidine, and piperidine.

As discussed previously, dimeric products are also of interest, and would likewise be investigated (FIG. L). In direct analogy to the preparation of 38, reaction of 35 with diamines such as 40 would lead to dimeric products 41. Use of N,N′-dimethylaminoalkanes 18 would lead to quaternary salts 42, which could also be prepared by methylation of 41.

There are a number of known non-peptidic compounds that bind to endothelin receptors, a few of which are shown below in FIG. M.

For example, Ro-46-2005 was the first orally active non-peptidic antagonist of the endothelin receptor, while a close analog, bosentan, was found to be even more active. Isoxazole BMS-1 82874 was also found to be active, though a closely related compound possessing an N-methylsulfonamide showed little binding ability, suggesting that the sulfonamide hydrogen was necessary for efficient receptor binding. A number of these compounds have been found to be active at very low concentrations. For example, sulfonamide 43 has been found to have an IC₅₀ of only 0.55 nM (1).

In addition to sulfonamide-based inhibitors, a number of carboxylic acid derivatives have also shown good activity. For example, darusentan and its analog 44 are both effective endothelin receptor antagonists. Similarly, SB-209670 shows affinity for both ETB and ETA receptors at nanomolar and subnanomolar concentrations, respectively.

Looking at the structures shown in FIG. M, one can see that a feature common to them all is the presence of an acidic proton (either carboxylic acid or sulfonamide) located a few angstroms away from two or three aromatic rings, at least one of which is electron-rich. Thus, in order to facilitate binding of PM 2010 analogs to the endothelin receptor, we propose appending such functionality onto the tanshinone IIA core. One method of achieving this is shown in FIG. N.

Starting from sulfonic acid 33 (prepared as shown earlier in FIG. I), treatment with PCI₅ would yield sulfonyl chloride 45. This could then be treated with a variety of readily available amines to yield the corresponding sulfonamide derivatives, a few representative examples of which are shown above. This approach allows the introduction of a wide variety of amine substituents, with once again preliminary results guiding further elaboration of 45.

Alternatively, variations in the sulfonyl moiety could be investigated. It has been shown that treatment of furans with the Vilsmeier reagent (DMF/POCI₃), followed by hydroxylamine, then Raney nickel, provides ready access to the corresponding aminomethyl furans (4). Applying this protocol to 8 (PM 2010) would be expected to yield amine 50 (FIG. O). Treatment of 14 (prepared as shown in FIG. D) with an excess of ammonia would also be expected to produce 50. Reaction of 50 with a variety of sulfonyl chlorides would provide access to a number of sulfonamides such as 51-53.

Derivatives possessing a carboxylic acid group could also be made starting from 14. For example, treatment of 14 with the dianion of malic acid would be expected to yield phenylacetic acid derivative 54 after acidic work-up. Once again, this is simply one example of a series of compounds which could be prepared in this manner.

An alternative approach to carboxylic acid derivatives is shown in FIG. P. Lithiation of C-2 of the furan ring would yield intermediate 55, which could be trapped by aldehydes or ketones to yield alcohol derivatives such as 56 and 57. As before, only two representative examples using commercially available carbonyl compounds are shown, though an extensive series of derivatives could be prepared in an analogous manner. The carboxylic acid functionality could be introduced by alkylation of the alcohols with sodium chloroacetate yielding compounds such as 58 and 59. Further variations could be introduced by changing the nature of the alkylating agent.

### REFERENCES

1. Makarov S S. “NF-κB as a therapeutic target in chronic inflammation: recent advances.” Mol Med Today 6:441-8 (2000).
2. Pahl H L. “Activators and target genes of Rel/NF-κB transcription factors.” Oncogene 18:6953-66 (1999).
3. Baldwin A S, Jr. “The NF-κB and I-κB proteins: new discoveries and insights.” Annu. Rev. Immunol. 14:649-83 (1996).
4. Ghosh S, May M J, Kopp E B. “NF-κB and Rel proteins: evolutionarily conserved mediators of immune responses.” Annu. Rev. Immunol. 16:225-60 (1998).
5. Whiteside S T, Israel A. “I-κB proteins: structure, function and regulation.” Semin. Cancer Biol. 8:75-82 (1997).
6. Karin M. Ben-Neriah Y. “Phosphorylation meets ubiquitination: the control of NF-κB activity.” Annu. Rev. Immunol. 18:621-663 (2000).
7. Israel A. “The IKK complex: an integrator of all signals that activate NF-.kappa.B.” Trends Cell Biol. 10:129-33 (2000).
8. Rothwarf D M, Zandi E, Natoli G, Karin M. “IKK-.gamma. is an essential regulatory subunit of the I-κB kinase complex.” Nature 395:297-300 (1998).
9. Yamaoka S, Courtois G, Bessia C, Whiteside S T, Weil R, Agou F, Kirk H E, Kay R J, Israel A. “Complementation cloning of NEMO, a component of the I-κB kinase complex essential for NF-κB activation.” Cell 93:1231-40 (1998).
10. Rayet B, Gelinas C. “Aberrant rel/nfkb genes and activity in human cancer.” Oncogene 18:6938-47 (1999).
11. Mayo M W, Badwin A S. “The transcription factor NF-.kappa.B: control of oncogenesis and cancer therapy resistance.” Biochim. Biophys. Acta 1470:M55-M62 (2000).
12. Romashkova J A, Makarov S S. “NF-κB is a target of AKT in anti-apoptotic PDGF signaling.” Nature 401:86-90 (1999).
13. Hinz M, Krappmann D, Eichten A, Heder A, Scheidereit C, Strauss M. “NF-κB function in growth control: regulation of cyclin Dl expression and G.sub.0/G.sub.1-to-S-phase transition.” Mol. Cell. Biol. 19:2690-8 (1999).
14. Barkett M, Gilmore T D. “Control of apoptosis by Rel/NF-κB transcription factors.” Oncogene 18:6910-24 (1999).
15. Gasparian A V, Yao Y J, Lu J, Yemelyanov A Y, Lyakh L A, Slaga T J, Budunova I V. “Selenium compounds inhibit I-κB kinase (IKK) and nuclear factor-.kappa.B (NF-.kappa.B) in prostate cancer cells.” Mol Cancer Ther. 1:1079-87 (2002).
16. Yamamoto Y, Gaynor R B. “Therapeutic potential of inhibition of the NF-κB pathway in the treatment of inflammation and cancer.” J. Clin. Invest. 107135-42 (2001).
17. Shen W, Waldschmidt M, Zhao X, Ratliff T, Krieg A M. “Antitumor mechanisms of oligodeoxynucleotides with CpG and polyG motifs in murine prostate cancer cells: decrease of NF-κB and AP-1 binding activities and induction of apoptosis.” Antisense Nucleic Acid Drug Dev. 12:155-64 (2002).
18. Karin M. “New twists in gene regulation by glucocorticoid receptor: is DNA binding dispensable?” Cell 93:487-90 (1998).
19. Yang J P, Merin J P, Nakano T, Kato T, Kitade Y, Okamoto T. “Inhibition of the DNA-binding activity of NF-κB by gold compounds in vitro.” FEBS Lett. 361:89-96 (1995).
20. Yin M J, Yamamoto Y, Gaynor R B. “The anti-inflammatory agents aspirin and salicylate inhibit the activity of I-κB kinase-.beta..” Nature 396:77-80 (1998).
21. Epinat J C, Gilmore T D. “Diverse agents act at multiple levels to inhibit the Rel/NF-κB signal transduction pathway.” Oncogene 18:6896-6909 (1999).
22. Jobin C, Bradham C A, Russo M P, Juma B, Narula A S, Brenner D A, Sartor R B. “Curcumin blocks cytokine-mediated NF-κB activation and proinflammatory gene expression by inhibiting inhibitory factor I-.kappa.B kinase activity.” J. Immunol. 163:3474-83 (1999).
23. Tomita T, Takeuchi E, Tomita N, Morishita R, Kaneko M, Yamamoto K, Nakase T, Seki H, Kato K, Kaneda Y, Ochi T. “Suppressed severity of collagen-induced arthritis by in vivo transfection of nuclear factor kappa B decoy oligodeoxynucleotides as a gene therapy.” Arthritis Rheum. 42:2532-42 (1999).
24. Bantel H, Schmitz M L, Raible A, Gregor M, Schulze-Osthoff K. “Critical role of NF-κB and stress-activated protein kinases in steroid unresponsiveness.” FASEB J. 16:1832-34 (2002).
25. Kaltschmidt B, Uherek M, Wellmann H, Volk B, Katschmidt C. “Inhibition of NF-κB potentiates amyloid beta-mediated neuronal apoptosis.” Proc. Natl. Acad. Sci., U.S.A. 96:9409-14 (1999).
26. Bates P J, Kahlon J B, Thomas S D, Trent J O, Miller D M “Antiproliferative activity of G-rich oligonucleotides correlates with protein binding.” J. Biol. Chem. 274:26369-77 (1999).
27. Derenzini M, Sirri V, Trere D, Ochs R L. “The quantity of nucleolar proteins nucleolin and protein B23 is related to cell doubling time in human cancer cells.” Lab. Invest. 73:497-502 (1995).
28. Roussel P, Sirri V, Hernandez-Verdun D. “Quantification of Ag-NOR proteins using Ag-NOR staining on western blots.” J. Histochem Cytochem. 42:1513-7 (1994).
29. Pich A, Chiusa L, Margaria E. “Prognostic relevance of AgNORs in tumor pathology.” Micron. 31:133-41 (2000).
30. Trere D, Derenzini M, Sirri V, Montanaro L, Grigioni W, Faa G, Columbano G M, Columbano A. “Qualitative and quantitative analysis of AgNOR proteins in chemically induced rat liver carcinogenesis.” Hepatology 24:1269-73 (1996).
31. Data derived from genome.ucsc.edu (to locate nucleolin sequence at 2q37.1) and the Mitelman Database of Chromosome Aberrations in Cancer (cgap.nci.nih.gov/Chromosomes).
32. Srivastava M, Pollard H B. “Molecular dissection of nucleolin's role in growth and cell proliferation: new insights.” FASEB J. 13:1911-22 (1999).
33. Tuteja R, Tuteja N. “Nucleolin: a multifunctional major nucleolar phosphoprotein.” Crit. Rev. Biochem. Mol. Biol. 33:407-36 (1998).
34. Ginisty H, Sicard H, Roger B, Bouvet P. “Structure and functions of nucleolin.” J. Cell. Sci. 112 (Pt 6):761-72 (1999).
35. Yang C, Maiguel D A, Carrier F. “Identification of nucleolin and nucleophosmin as genotoxic stressresponsive RNA-binding proteins.” Nucleic Acids Res. 30:2251-60 (2002).
36. Daniely Y, Borowiec J A. “Formation of a complex between nucleolin and replication protein A after cell stress prevents initiation of DNA replication.” J. Cell Biol. 149:799-810 (2000).
37. Wang Y, Guan J, Wang H, Wang Y, Leeper D, lliakis G. “Regulation of dna replication after heat shock by replication protein a-nucleolin interactions.” J. Biol. Chem. 276:20579-88 (2001).
38. Bates P J, Miller D M, Trent J 0, Xu, X. “Method for the diagnosis and prognosis of malignant diseases,” U.S. patent application Ser. No. 10/118,854, filed in the USPTO on Apr. 8, 2002.
39. Barel M, Le Romancer M, Frade R. “Activation of the EBV/C3d receptor (CR2, CD21) on human B lymphocyte surface triggers tyrosine phosphorylation of the 95-kDa nucleolin and its interaction with phosphatidylinositol 3 kinase.” J. Immunol. 166:3167-73 (2001).
40. Larrucea S, Cambronero R, Gonzalez-Rubio C, Fraile B, Gamallo C, Fontan G, Lopez-Trascasa M. “Internalization of factor J and cellular signalization after factor J-cell interaction.” Biochem. Biophys. Res. Commun. 266:51-7 (1999).
41. Dumler I, Stepanova V, Jerke U, Mayboroda O A, Vogel F, Bouvet P, Tkachuk V, Haller H, Gulba D C. “Urokinase-induced mitogenesis is mediated by casein kinase 2 and nucleolin.” Curr. Biol. 9:1468-76 (1999).
42. Hovanessian A G, Puvion-Dutilleul F, Nisole S, Svab J, Perret E, Deng J S, Krust B. “The cell-surface expressed nucleolin is associated with the actin cytoskeleton.” Exp. Cell Res. 261:312-28 (2000).
43. Callebaut C, Blanco J, Benkirane N, Krust B, Jacotot E, Guichard G, Seddiki N, Svab J, Dam E, Muller S, Briand J P, Hovanessian A G. “Identification of V3 loop-binding proteins as potential receptors implicated in the binding of HIV particles to CD4(+) cells.” J. Biol. Chem. 273:21988-97 (1998).
44. Borer R A, Lehner C F, Eppenberger H M, Nigg E A. “Major nucleolar proteins shuttle between nucleus and cytoplasm.” Cell 56:379-90 (1989).
45. Martin P, Duran A, Minguet S, Gaspar M L, Diaz-Meco M T, Rennert P, Leitges M, Moscat J. “Role of zeta PKC in B-cell signaling and function.” EMBO J. 21:4049-57 (2002).
46. Zhou G, Seibenhener M L, Wooten M W. Nucleolin is a protein kinase C-zeta substrate. Connection between cell surface signaling and nucleus in PC12 cells.” J. Biol. Chem. 272:31130-7 (1997).
47. Turutin D V, Kubareva E A, Pushkareva M A, Ullrich V, Sud'ina G F. “Activation of NF-kappa B transcription factor in human neutrophils by sulphatides and L-selectin cross-linking.” FEBS Lett. 536:241-5 (2003).
48. Harms G, Kraft R, Grelle G, Volz B, Dernedde J, Tauber R. “Identification of nucleolin as a new L selectin ligand.” Biochem. J. 360(Pt 3):531-8 (2001).
49. Salazar R, Brandt R, Krantz S. “Binding of Amadori glucose-modified albumin by the monocytic cell line MonoMac 6 activates protein kinase C epsilon. protein tyrosine kinases and the transcription factors AP-1 and NF-.kappa.B.” Glycoconj J. 18:769-77 (2001).
50. Sugano N, Chen W, Roberts M L, Cooper N R. “Epstein-Barr virus binding to CD21 activates the initial viral promoter via NF-κB induction.” J. Exp Med. 186:731-7 (1997).
51. Gil D, Gutierrez D, Alarcon B. “Intracellular redistribution of nucleolin upon interaction with the CD3.epsilon. chain of the T cell receptor complex.” J. Biol. Chem. 276:11174-9 (2001).
52. Weil R, Schwamborn K, Alcover A, Bessia C, Di Bartolo V, Israel A. “Induction of the NF-κB cascade by recruitment of the scaffold molecule NEMO to the T cell receptor.” Immunity 18:13-26 (2003).
53. Miranda G A, Chokler I, Aguilera R J. “The murine nucleolin protein is an inducible DNA and ATP binding protein which is readily detected in nuclear extracts of lipopolysaccharide-treated splenocytes.” Exp. Cell Res. 217:294-308 (1995).
54. Hauser H, Gains N. Spontaneous vesiculation of phospholipids: a simple and quick method of forming unilamellar vesicles. Proc. Natl. Acad. Sci., U.S.A. 79:1683-7 (1982).
55. Pitcher W H III, Huestis W H. “Preparation and analysis of small unilamellar phospholipid vesicles of a uniform size.” Biochem. Biophys. Res. Commun. 296:1352-5 (2002).
56. Wang H, Yu D, Agrawal S, Zhang R. “Experimental therapy of human prostate cancer by inhibiting MDM2 expression with novel mixed-backbone antisense oligonucleotides: In vitro and in vivo activities and mechanisms.” Prostate 54:194-205 (2003).
57. Chi K N, Gleave M E, Klasa R, Murray N, Bryce C, Lopes de Menezes D E, D'Aloisio S, Tolcher A W. “A phase I dose-finding study of combined treatment with an antisense Bcl-2 oligonucleotide (Genasense) and mitoxantrone in patients with metastatic hormone-refractory prostate cancer.” Clin. Cancer Res. 7, 3920-3927 (2001).
58. Coqueret 0, Gascan H. “Functional interaction of STAT3 transcription factor with the cell cycle inhibitor p21WAF1/CIP1/SDI1.” J. Biol. Chem. 275:18794-800 (2000).
59. Jensen 0, Wilm M, Shevchenko A, Mann M. “Peptide sequencing of 2-DE gel-isolated proteins by nanoelectrospray tandem mass spectrometry.” Methods Mol. Biol. 112:513-30 (1999).
60. Kikuchi E, Horiguchi Y, Nakashima J, Kuroda K, Oya M, Ohigashi T, Takahashi N, Shima Y, Umezawa K, Murai M. “Suppression of hormone-refractory prostate cancer by a novel nuclear factor KB inhibitor in nude mice.” Cancer Res. 63:107-10 (2003).
61. Nabel, E G, Nabel, G J. “Treatment of diseases by site-specific instillation of cells or site-specific transformation of cells and kits therefore.” U.S. Pat. No. 5,328,470. (1994).
62. Chen, S. H., H. D. Shine, J. C. Goodman, R. G. Grossman, et al. 1994. Gene therapy for brain tumors: regression of experimental gliomas by adenovirus-mediated gene transfer in vivo. Proc Natl Acad Sci USA. 91:3054-7.

What is claimed is:

1. A composition of the formula 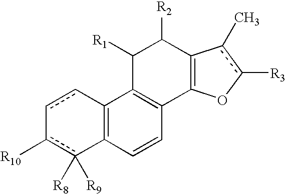
wherein R₁ and R₂ each being the same or different are O or hydroxyl or wherein R₁ and R₂ are taken together to form a 5 member ring of the formula: 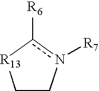
wherein R₆ and R₇ each being the same or different are hydrogen or alkyl of 1 to 6 carbon atoms; wherein R₃ is hydrogen, (CH3)₂N(alkyl of 1 to 6 carbon atoms), R₄—N—R₄—N—R₅, SO₂R₁₁, or a composition of the formula: 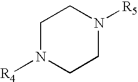
wherein R₄ is alkyl of 1 to 6 carbon atoms, R₅ is hydrogen alkyl of 1 to 6 carbon atoms, or methyl tashione IIA and R₁₁ is hydroxyl, diphenyl-NH, alkoxy of 1 to 6 carbon atom phenyl R₁₂—NH, or phenyl substituted phenyl (R₁₂)—NH and wherein R₁₂ is alkyl of 0 to 6 carbon atoms, phenyl substituted alkyl or alkyl of 1 to 6 carbon atoms-COOH;
wherein R₈, R₉ and R₁₀ separately or together is hydrogen, alkyl of 1 to 6 carbon atoms or haloalkyl of 1 to 6 carbon atoms; or wherein R₈ and R₉ are taken together to for a ring of the formula: 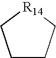
wherein R₁₃ and R₁₄ each being the same or different are 0, N Alkyl of 1-6 carbon atoms-N, Dialklyl of 1-6 carbon atoms-N or (alkyl of 1 to 6 carbon atoms)—CO₂-(alkyl of 1 to 6 carbon atoms) wherein when R₃ is hydrogen R₁ and R₂ can not both be O.

2. A composition according to claim 1 wherein the composition is used to treat a disease selected from the group consisting of inflammatory and tissue repair disorders, particularly rheumatoid arthritis, inflammatory bowel disease, asthma and chronic obstructive pulmonary disease, osteoarthritis, osteoporosis and fibrotic diseases, dermatosis, including psoriasis, atopic dermatitis and ultraviolet radiation -induced skin damage, autoimmune diseases including systemic lupus eythematosus, multiple sclerosis, psoriatic arthritis, alkylosing spondylitis, tissue and organ rejection, Alzheimer's disease, stroke, atherosclerosis, restenosis, diabetes, glomerulonephritis, cancer, including Hodgkins disease, cachexia, inflammation associated with infection and certain viral infections, including aquired immune deficiency syndrome, adult respiratory distress syndrome, and Ataxia Telangiestasia.
